(19) United States
(12) Patent Application Publication
(10) Pub. No.: US 2023/0414552 A1
(43) Pub. Date: Dec. 28, 2023

(54) NOVEL METHODS

(71) Applicants: Colgate-Palmolive Company, New York, NY (US); The Trustees of the University of Pennsylvania, Philadelphia, PA (US)

(72) Inventors: Payal ARORA, Lebanon, NJ (US); Chider CHEN, Philadelphia, PA (US)

(21) Appl. No.: 18/266,096

(22) PCT Filed: Dec. 10, 2021
(86) PCT No.: PCT/US21/62939

§ 371 Date: Jun. 8, 2023

Related U.S. Application Data
(60) Provisional application No. 63/123,880, filed on Dec. 10, 2020.

(51) Int. Cl. A61K 31/26; G01N 33/50; A61P 1/02; A61K 31/05; A61K 31/12; A61K 31/19

(52) U.S. Cl. CPC A61K 31/26; G01N 33/5073; G01N 33/5023; A61P 1/02; A61K 31/05; A61K 31/12; A61K 31/19

(57) ABSTRACT

The invention relates to methods of treatment and screening natural compounds that can be used to further proliferate orally derived stem cells (e.g., periodontal ligament stem cells, dental pulp stem cells, or stem cells from the apical papilla or gingiva-derived mesenchymal stem cells (GMSC)).

### FIELD OF THE INVENTION

The invention relates to methods of treatment and screening compounds that can be then be used to further proliferate orally derived stem cells (e.g., periodontal ligament stem cells, stem cells from the apical papilla or gingiva-derived mesenchymal stem cells (GMSC)). The invention relates to the proliferation of orally derived stems that can be used to stimulate dental osteogenesis.

### BACKGROUND

Periodontitis is a relatively common chronic infection that can result in the destruction of tendon-like soft tissues and mandibular bones which support the teeth. As the remaining periodontal tissues have little regenerative ability, these injuries are can be difficult to manage due to the complex structure, activated inflammation, and fibrotic scar formation during regeneration of damaged tissues, leading to the formation of poor-quality tissue with low mechanical functional strength. There is believed to be a demand for the repair of periodontal defects. However, the treatments have several disadvantages. Some of the drawbacks may include: donor site morbidity, inflammation, hematomas, and high cost and unnecessary growth, non-specificity and unwanted side effects.

Mesenchymal stem cells (MSCs) constitute a population of self-renewal multipotent stem cells that can give rise to multiple specialized cell types, along with their extensive distribution in many adult tissues, have made them an attractive target for tissue engineering applications. MSCs derived from orofacial sources, such as periodontal ligament stem cells (PDLSCs) and gingival mesenchymal stem cells (GMSCs), have superior capability for tendon-like ligament tissue regeneration, which are very attractive for craniofacial applications, as they may be more committed to differentiating into orofacial tissues. Despite the applications of dental MSCs for the repair of orofacial defects used in animal models, the periodontal tissue regeneration often results in an unfavorable outcome due to the rapid apoptosis of transplanted MSCs.

The survival of implanted MSCs may be negatively affected by the recipient immune system, which challenges their tissue regeneration capacity. MSC-mediated bone regeneration is partially controlled by the host local microenvironment, including the presence of growth factors as well as host immune cells and cytokines.

It is desirable to develop treatment regimens that can stimulate osteogenesis that are relatively non-invasive. However, there are obstacles in developing these methods. First, there is a desired balance between controlled osteogenesis and uncontrolled growth. Finding compounds and treatment regimens to achieve the right amount of osteogenesis may be difficult. For example, some endogenous growth factors are ubiquitous, unstable, and their signaling pathways are non-specific. This can make certain growth factors difficult targets. Second, while certain methods have attempted to use live-stem cell therapy, these regiments also have drawbacks in that the introduction of live stem cells may be rejected by the host.

There is an unmet need for periodontal tissue regeneration for preserving teeth and keeping periodontal tissue healthy and avoiding painful periodontal surgeries for patients with mild disease to severe disease with significant loss of tissue and bone.

### BRIEF SUMMARY

The inventors have surprisingly developed a method to treat patients with one or more compounds that modulate a molecular target in the ERK pathway. Without being bound by theory, the treatment with a compound that can modulate a target of the ERK pathway in the oral cavity can, in turn, stimulate periodontal ligament stem cells (PDLSCs) for osteogenesis and/or mineralization, and gingival mesenchymal stem cells (GMSCs) for soft tissue regeneration. In one aspect, the ERK modulating compounds are able to generate reparative periodontal tissue (e.g., periodontal ligament stem cells (PDLSCs)) that may be able to restore lost periodontium, i.e., cementum, alveolar bone, and periodontal ligament.

In one aspect, the compound can be administered to a patient that still has endogenous stem cells in the oral cavity (e.g., endogenous periodontal ligament stem cells). In one aspect, the treatment method can be administered to patients that still retain a certain amount of endogenous stem cell in the oral cavity (e.g., endogenous periodontal ligament stem cells), as well patients that have little to no existing endogenous stem cell in the oral cavity. In one aspect, patients that have little to no existing endogenous stem cell in their oral cavity can be treated with a combination therapy of a compound that modulates a molecular target of the ERK pathway, as well as live-stem cell therapy. Without being bound by theory, the advantage of providing a compound that modulates a molecular target of the ERK pathway is that it provides an immunomodulatory benefit to reduce the risk that the host rejects the live-stem cell treatment. Without being bound by theory, avoiding host rejection could be critical in more advanced patients where an ERK modulating compound is given in combination with live-stem cell treatment to promote osteogenesis.

In another aspect, the inventors have developed a method to identify naturally occurring ERK activating compound, using orally derived stem cells, that can further be used to proliferate endogenous stem cells in the oral cavity.

Briefly, in healthy cells, and without being bound by theory, it is believed that the ERK pathway is activated as follows. RAS receptor-ligand binding results in cytoplasmic BRAF protein being localized to the intracellular membrane surface by binding directly to RAS (Jaiswal et al., Mol Cell Biol. 14(10):6944-53 (1994), the contents of which are incorporated by reference), which, in turn, results in BRAF phosphorylation. Once phosphorylated, BRAF serine/threonine kinase activity is activated and the activated enzyme phosphorylates MEK, which is also referred to as MAPKK. MEK phosphorylation, in turn, activates its kinase activity, and it in turn phosphorylates ERK, which is also referred to as MAPK. Upon phosphorylation, ERK is translocated into the nucleus, where it phosphorylates transcription factors and thereby stimulates transcription of various genes involved in cell growth, differentiation and apoptosis (Peyssonnaux et al, Biol Cell. 93 (1-2)-.53-62 (2001), the contents of which are incorporated by reference). One of the challenges of compounds that modulate the ERK pathway is that the pathway is ubiquitous and the molecular targets may be non-specific.

The pathway includes many other proteins, next to the above-mentioned MAPK (originally called ERK), BRAF and MEK (MAPKK). The latter are examples of proteins that can communicate by adding phosphate groups to a neighboring protein, which acts as an “on” or “off switch in the MAPK/ERK pathway. The present disclosure provides for methods that utilize natural compounds that are able to activate this pathway in order stimulate the growth of endogenous oral stem cells, for example endogenous periodontal ligament stem cells (“PDL stem cells”) or gingiva-derived mesenchymal stem cells (GMSC).

Cytokines, growth factors and/or differentiation that are affected by ERK pathway modulation can be potential targets as well in order to increase osteogenesis. In one aspect, this includes, but it not limited to stromal cell derived factor-1 (SDF-1), stem cell factor (SCF), angiopoietin-1, placenta-derived growth factor (PIGF), granulocyte-colony stimulating factor (G-CSF), any agent which promotes the expression of endothelial adhesion molecules, such as ICAMs and VCAMs, any agent which facilitates the homing process, vascular endothelial growth factor (VEGF), fibroblast growth factors (e.g., FGF4, FGF8, bFGF), Wnt11, DKK1, ascorbic acid, isoproterenol, endothelin, any agent which promotes angiogenesis, including VEGF, aFGF, angiogenin, angiotensin-1 and -2, betacellulin, bFGF, Factor X and Xa,

Without being bound by theory, the methods contemplated herein (e.g., any of Method 1.0 et seq) can be used to treat two different patient populations. In one aspect, natural compounds that function as ERK activators can be used to treat patients with moderate periodontitis with periodontal ligament stimulation for osteogenic differentiation and gingival mesenchymal stem cell stimulation for soft tissue regeneration. In another aspect, and without being bound by theory, natural compounds that act as ERK activators can be used in combination with periodontal ligament stem cells for patients with advanced (chronic) disease stage periodontitis who have severe tissue and dental bone loss. Without being bound by theory, the combination of ERK activation and immunomodulation, for example, can make these molecules ideal candidate for stem cells regeneration, and, in turn, increased dental osteogenesis.

In one aspect, the invention contemplates an assay is that it can it can be used to screen for compounds that stimulate existing orally derived stem cells.

In another aspect, the invention relates to the ability to screen for naturally occurring compounds that are then able to be applied to endogenous periodontal ligament stem cells (“PDL stem cells”) or gingiva-derived mesenchymal stem cells (GMSC) in the patient's oral cavity to generate reparative periodontal tissue that may be able to restore lost periodontium, i.e., cementum, alveolar bone, and periodontal ligament. This, in turn, can have practical value because it allows a dental or medical practitioner to provide better care to a patient in need of such treatment. For example, compounds identified by the current method could potentially be used to treat various periodontal diseases and trauma in a minimally invasive manner.

A number of methods in the art are directed to implanting or injecting exogenous stem cells into the oral cavity, which, in turn, can be invasive. Without being bound by theory, one drawback to this type of procedure in the art is that it can provoke a native T-cell response that can actually damage or kill the exogenous stem cells that are applied to the area.

However, in one aspect, a further advantage of the current method is that natural compounds that are identified can be used in place of exogenous stem cells in order to stimulate existing endogenous stem cells that are already present in the oral cavity. In one aspect, the disclosure provides for methods where natural compounds can be used to stimulate endogenous periodontal ligament stem cells. Without being bound by theory, stimulation of the periodontal ligament can be a less invasive means to stimulate stem cells in the oral cavity, as compared to some methods which rely on stimulation of the dental pulp. For example, the dental pulp may be more difficult to stimulate because it is surrounded by dentin, enamel, and potentially alveolar bone. Alternatively, the periodontal ligament may be less obstructed and easier to treat.

In one aspect the invention relates to a method of treatment of gingiva and/or periodontal ligament tissue to increase dental osteogenesis, mineralization or soft tissue regeneration in the oral cavity (Method 1.0), wherein the method comprises administering an effective amount of one or more natural ERK modulating compounds to the oral cavity of a patient in need thereof, wherein the patient has existing endogenous stem cells that are present in the oral cavity, and wherein the natural ERK modulating compound is administered to the endogenous stem cells (e.g., endogenous periodontal ligament stem cells or gingiva-derived mesenchymal stem cells).

In another aspect, Method 1.0 also contemplates the following:
1.1 The method of Method 1.0, wherein the natural ERK modulating compound is administered to endogenous periodontal ligament stem cells in the patient's oral cavity.
1.2 The method of Method 1.0 or 1.1, wherein the natural ERK modulating is administered in an amount effective to increase proliferation of endogenous periodontal ligament (PDL) stem cells or gingiva-derived mesenchymal stem cells (GMSC) in the patient's oral cavity.
1.3 The method of any of the preceding methods, wherein the natural compound modulates (e.g., increase) gene expression (e.g., mRNA) or protein expression of a component of the ERK/MAP kinase pathway.
1.4 The method of any of the preceding methods, wherein the natural ERK modulating compound can modulate (e.g., increase or decrease) the level of expression (e.g., mRNA or protein expression) of one or more of the following: cellular alkaline phosphatase (ALP), Runx2, bone marrow stromal cells, CD166, CD90, CD105, Stro-1, ATF4, LRP5, TGFβ, osteopontin (OPN), FAS, FASL, and osteocalcin (OCN).
1.5 The method of any of the preceding methods wherein the natural ERK modulating compound is administered to a patient with decreased expression levels, relative to a reference standard, (e.g., measured by mRNA or protein expression) of one or more of the following biomarkers: cellular alkaline phosphatase (ALP), Runx2, bone marrow stromal cells, CD166, CD90, CD105, Stro-1, ATF4, LRP5, TGFβ, osteopontin (OPN), FAS, FASL, and osteocalcin (OCN).
1.6 The method of any of the preceding methods, wherein the natural ERK modulating compound comprises a stilbenoid (e.g., a plant polyphenol).
1.7 The method of any of the preceding methods, wherein the natural ERK modulating compound comprises a pentacylic terpenoid.
1.8 The method of any of the preceding methods, wherein the natural ERK modulating compound comprises a curcuminoid (e.g., a diarylheptanoid) or is a compound that contains curcuminoids (e.g., turmeric).
1.9 The method of any of the preceding methods, wherein the natural ERK modulating compound comprises an isothiocyanate.
1.10 The method of any of methods 1.6-1.9, wherein the natural ERK modulating compound is selected from the group consisting of: resveratrol, boswellic acid, curcumin and Phenethyl isothiocyanate (PEITC)
1.11 The method of any of the preceding methods, wherein the natural compound comprises resveratrol.
1.12 The method of any of the preceding methods, wherein the natural compound comprises boswellic acid.
1.13 The method of any of the preceding methods, wherein the natural compound comprises Phenethyl isothiocyanate (PEITC).
1.14 The method of any of the preceding methods, wherein the natural compound comprises curcumin or an extract of curcumin (e.g., yellow curcumin extract and/or white curcumin extract).
1.15 The method of any of the preceding methods, wherein the amount of the natural compound is effective to increase the proliferation of endogenous PDL stem cells (e.g., from 0.1%-5% by wt.).
1.16 The method of any of the preceding methods, wherein the patient is at elevated risk, relative to a reference standard, of periodontal ligament stem cell damage and/or GMSC damage.
1.17 The method of any of the preceding methods, wherein the patient is at elevated risk, relative to a reference standard, of periodontal ligament tissue damage, and/or gingiva tissue damage.
1.18 The method of any of the preceding methods, wherein the patient has retained from 5%-75% of their existing endogenous periodontal ligament stem cells.
1.19 The method of any of the preceding methods, wherein the natural ERK modulating compound is administered to endogenous gingiva-derived mesenchymal stem cells (GMSC) in the patient's oral cavity.
1.20 The method of any of the preceding methods, wherein the natural ERK modulating compound is given in combination with live stem cell therapy.
1.21 The method of any of the preceding methods, wherein the natural ERK modulating compound modulates a cytokine or growth factor selected from the group consisting of: stromal cell derived factor-1 (SDF-1), stem cell factor (SCF), angiopoietin-1, placenta-derived growth factor (PIGF), granulocyte-colony stimulating factor (G-CSF), any agent which promotes the expression of endothelial adhesion molecules, e.g., ICAMs and VCAMs, any agent which facilitates the homing process, vascular endothelial growth factor (VEGF), fibroblast growth factors (e.g., FGF4, FGF8, bFGF), Wnt11, DKK1, ascorbic acid, isoproterenol, endothelin, any agent which promotes angiogenesis, e.g., VEGF, aFGF, angiogenin, angiotensin-1 and -2, betacellulin, bFGF, Factor X and Xa, and combinations thereof.
1.22 The method of any of the preceding methods, wherein the increased proliferation of periodontal ligament stem cells results in increased dental osteogenesis in the oral cavity.
1.23 The method of any of the preceding methods, wherein the stimulation of periodontal ligament stem cells results in increased amounts of periodontium (e.g., cementum, alveolar bone, and periodontal ligament).
1.24 The method of any of the preceding methods, wherein the natural compounds that function as ERK activators can be used for treatment, in moderate periodontitis patients, wherein the natural compounds provide periodontal ligament stimulation for osteogenic differentiation and gingival mesenchymal stem cell stimulation for soft tissue regeneration.
1.25 The method of any of Method 1.0-1.23, wherein the natural compounds that act as ERK activators can be used for treatment, in combination with periodontal ligament stem cells, for advanced (chronic) disease stage periodontitis patients who have severe tissue and dental bone loss.
1.26 The method of any of the preceding methods, wherein the amount of the natural compound administered is from 0.5 μM to 50 μM (e.g, about 1 μM) (e.g., about 5 μM).
1.27 The method of any of the preceding methods, wherein the natural ERK modulating compound(s) is/are administered orally, topically (e.g., on the gums), injection, implantation, or any combination thereof.
1.28 The method of any of the preceding methods, wherein the natural ERK modulating compound(s) is/are administered in a form selected from the following: tablets, capsules, solutions, suspensions, and combinations thereof.

In one aspect, the disclosure contemplates any of the natural ERK modulating compounds referenced herein, for use in any of the methods, e.g., any of Method 1.0 et seq., described herein. In another aspect, any of the natural ERK modulating compounds referenced herein, e.g., any of Method 1.0 et seq, may exist in free or in orally acceptable salt form. In yet another aspect, the methods, e.g., any of Method 1.0 et seq, comprise the use of natural ERK modulating compounds in combination or for co-administration with other active agents.

In another aspect, the patient of any of Method 1.0 et seq, has no patient has no remaining endogenous stem cells in the oral cavity (e.g., no remaining endogenous periodontal ligament stem cells). In yet another aspect, an ERK modulating compound of any of Method 1.0 et seq is administered to a patient with no endogenous stem cells in the oral cavity (e.g., no remaining endogenous periodontal ligament stem cells) in combination with live stem cell therapy.

In one aspect the invention relates to a screening method to detect natural ERK activating compounds that can stimulate endogenous oral stem cells, for example, periodontal ligament stem cell or gingiva-derived mesenchymal stem cells (GMSC), proliferation in the oral cavity (Method 2.0). Method 2.0 is a method of selecting natural ERK activating compounds that cause the proliferation of endogenous oral stem cells (e.g., dental pulp stem cells, periodontal ligament stem cells, gingiva-derived mesenchymal stem cells (GMSC), or stem cells from the apical papilla) wherein the method comprises:
(1) identifying natural ERK activating test compounds by observing increasing in mineralization relative to and a negative control, and/or gene expression of osteogenic biomarkers relative to a positive control (e.g., t-Butylhydroquinone) and a negative control;
(2) isolating periodontal ligament stem cell cells and/or or gingiva-derived mesenchymal stem cells from an oral cavity;
(3) identifying periodontal ligament stem cells and/or or gingiva-derived mesenchymal stem cells;
(4) contacting and screening periodontal ligament stem cells and/or or gingiva-derived mesenchymal stem cells with identified natural ERK activating test compounds;
(5) determining whether a test compound results in the proliferation of periodontal ligament stem cells and/or or gingiva-derived mesenchymal stem cells; and
(6) selecting a test compound for further development on the basis of whether it increases proliferation of periodontal ligament stem cells and/or or gingiva-derived mesenchymal stem cells relative to a standard control.

In another aspect, Method 2.0 also contemplates the following:
2.1 The Method of 2.0, wherein the method is performed as part of a high-throughput method (e.g., in a 96-well format).
2.2 The Method of 2.0 or 2.1, wherein the test compound is tested again t-Butylhydroquinone to observe whether the lead compound can increase mineralization.
2.3 The Method of 2.2, wherein the gene expression or protein expression of a particular cellular marker for osteogenesis is evaluated.
2.4 The Method of 2.3, wherein the level (e.g., mRNA or protein expression) of one or more of the following biomarkers is tested: cellular alkaline phosphatase (ALP), Runx2, bone marrow stromal cells, CD166, CD90, CD105, Stro-1, ATF4, LRP5, TGFβ, osteopontin (OPN), FAS, FASL, and osteocalcin (OCN).
2.5 Any of the preceding methods, wherein the chemical structure of the natural test compound is chemically altered (e.g., so that it can be stable in formulation).
2.6 Any of the preceding methods, determining the effect of the natural test compound of interest on bone formation and mineralization in cell culture (in vitro).
2.7 Any of the preceding methods, determining the effect of the natural test compound of interest on bone formation and mineralization in a non-human mammal (in vivo).
2.8 The method of 2.7, wherein the non-human mammal is a mouse.
2.9 Any of the preceding methods, wherein the bone formation and mineralization of the test compound is determined by measuring trabecular number, thickness, and/or spacing.
2.10 Any of the preceding methods, wherein the bone formation and mineralization of the test compound is determined by measuring bone volume, and/or measuring volumetric bone mineral density.
2.11 Any of the preceding methods, wherein the PDL stem cells are identified by flow cytometry or ELISA.
2.12 Any of the preceding methods, wherein PDL stem cells or gingiva-derived mesenchymal stem cells can be maintained is culture media selected from: Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®.
2.13 Any of the preceding methods, wherein a test compound is selected for further development because it increased PDL proliferation and/or or gingiva-derived mesenchymal stem cells in cell culture.
2.14 Any of the preceding methods, wherein a test compound is selected for further development because it increased PDL proliferation and/or or gingiva-derived mesenchymal stem cells in an animal model.
2.15 Any of the preceding methods, wherein a test compound is identified by its ability to modulate the gene expression (e.g., mRNA) or protein expression of a molecular target or biomarker of the ERK pathway.
2.16 The method of 2.15, wherein the biomarker is selected from: RSK, MNK, eIF4E, JUN, Fos, SRF, CREB, ATF1, Histone H3, HMG-14, Elk-1, Myc, Max, BRF1, UBF, PP1, PP2A, Akt, Raf, Raf inhibitors, Src, and PAK.
2.17 The method of any of the preceding methods, wherein the molecular target of the ERK pathway further regulates WNT signaling.
2.18 The method of any of the preceding methods, wherein the natural compound selected for further development is selected from the group consisting of: resveratrol, boswellic acid, curcumin or an extract of curcumin, and Phenethyl isothiocyanate (PEITC).
2.19 The method of any of the preceding methods, wherein the natural compound increases dental osteogenesis.
2.20 The method of any of the preceding methods, wherein the natural compound selected for further development can be used to increase proliferation of periodontal ligament stem cells and/or or gingiva-derived mesenchymal stem cells in a patient with periodontitis (e.g., moderate or chronic).

In another aspect, the Methods of Method 2.0 et seq, can be used to identify compounds that cause proliferation of dental pulp stem cells as well as apical papilla stem cells.

In one aspect, the methods described herein contemplate the use of Runx2 as a marker of bone mineralization increase. The transcription factor Runx2 is believed to be involved in osteoblast differentiation during embryonic development and to interact with a number of nuclear transcription factors, coactivators, and adaptor proteins that interpret extracellular signals to control homeostatic osteoblast development and activity (Lian, J. B., et al. (2004). Crit Rev Eukaryot Gene Expr 14, 1-41; Stein, G. S., et al. (2004). Oncogene 23, 4315-4329, the contents of which are incorporated herein by reference).

In one aspect, the methods described herein contemplate the use of the osteogenic biomarker ALP. In one aspect, bone-specific alkaline phosphatase (BAP) is synthesized by the osteoblasts and, without being bound by theory, is believed to be involved in the calcification of bone matrix.

### DETAILED DESCRIPTION

As used herein “Periodontal ligament (PDL)” is intended to refer to the soft, specialized connective tissue that connects the cementum of the tooth and to the alveolar bone of the maxillary and mandible to maintain teeth in situ, support teeth for function, and preserve tissue homeostasis. A “periodontal ligament stem cell” refers to a postnatal stem cell that is isolated from the periodontal ligament. As used herein, “Periodontal Ligament Stem Cells” refers to stem cells isolated from periodontal ligament that are capable of differentiating into a variety of cell types. These cell types may include, e.g., cementoblasts, cementocytes, adipocytes, and fibroblasts.

As used herein, “stem cell” refers to a relatively undifferentiated cell that can be induced to proliferate and that can produce progeny that subsequently differentiate into one or more mature cell types. In certain instances stem cells are “multipotent” because they can produce progeny of more than one distinct cell type.

As used herein, “differentiation” refers to the developmental process whereby cells assume a specialized phenotype, i.e., acquire one or more characteristics or functions distinct from other cell types.

The term “trauma” refers to an event that causes a cell to undergo a detrimental change. Examples of trauma include, physical injury resulting from accident or medical treatment, including surgery, disease (e.g., periodontal disease), degeneration, and the like.

As used herein, “subject” refers to any vertebrate, preferably a mammal, more preferably a human. Mammals include, but are not limited to, humans, farm animals, sport animals, and pets.

As used herein, “treat” or “treating” includes treating, preventing, ameliorating, or inhibiting physical or disease related damage and/or a symptom of physical or disease related damage of a subject.

As used herein, an “effective amount” generally means an amount which provides the desired local or systemic effect and performance. For example, an effective dose is an amount sufficient to affect a beneficial or desired clinical result.

As used herein, “reference standard” refers to prior measurement and obtaining of results in a control population.

As used herein, “Phenethyl Isothiocyanate” (PEITC) refers to an isothiocyanate found in cruciferous vegetables with chemopreventive and potential antitumor activities.

As used herein, “resveratrol” refers to 3,5,4′-trihydroxy-trans-stilbene which is a stilbenoid, a type of natural phenol, and a phytoalexin produced by several plants in response to injury or when the plant is under attack by pathogens, such as bacteria or fungi. Sources of resveratrol in food can include the skin of grapes, blueberries, raspberries, mulberries, and peanuts.

As used herein, “Boswellic acids” are a series of pentacyclic terpenoid molecules that are produced by plants in the genus Boswellia.

As used herein, “curcumin” refers to a bright yellow chemical produced by Curcuma longa plants. It is the principal curcuminoid of turmeric (Curcuma longa), a member of the ginger family, Zingiberaceae. It is sold as an herbal supplement, cosmetics ingredient, food flavoring, and food coloring. Chemically, curcumin is a diarylheptanoid, belonging to the group of curcuminoids, which are natural phenols responsible for turmeric's yellow color. It is a tautomeric compound existing in enolic form in organic solvents and in keto form in water. As used herein, “curcumin” is used interchangeable with “an extract of curcumin”.

### Cell Culture

In one aspect, e.g., any of Method 1.0 et seq or Method 2.0 et seq., PDLSCs can be maintained and grow in culture medium commercially available from the American Type Culture Collection (ATCC). Such media include, but are not limited to Dulbecco's Modified Eagle's Medium® (DMEM), DMEM F12 Medium®, Eagle's Minimum Essential Medium®, F-12K Medium®, Iscove's Modified Dulbecco's Medium®, RPMI-1640 Medium®.

Also contemplated is supplementation of cell culture medium with mammalian sera which can include, but are not limited to: fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements, and bovine embryonic fluid.

Additional supplements can also be added and include, but are not limited to: insulin, transferrin, sodium selenium and combinations thereof. These components can be included in a salt solution such as, but not limited to Hanks' Balanced Salt Solution® (HBSS), Earle's Salt Solution®, antioxidant supplements, MCDB-201® supplements, phosphate buffered saline (PBS), ascorbic acid and ascorbic acid-2-phosphate, as well as additional amino acids. Amino acid supplementation can also be included, for example: alanine, arginine, aspartic acid, asparagine, cysteine, cystine, glutamic acid, glutamine, glycine, histidine, isoleucine, L-leucine, lysine, methionine, phenylalanine, proline, serine, threonine, tryptophan, tyrosine, and valine.

Bacterial, mycoplasmal, and fungal contamination may addressed by antibiotics or anti-mycotic compounds, for example, mixtures of penicillin/streptomycin, including, but not limited to: amphotericin (Fungizone®), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones that may be used include: D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine. Additionally, the following cytokines and/or growth factors can also be used which include, but not limited to: stromal cell derived factor-1 (SDF-1), stem cell factor (SCF), angiopoietin-1, placenta-derived growth factor (PIGF), granulocyte-colony stimulating factor (G-CSF), any agent which promotes the expression of endothelial adhesion molecules, such as ICAMs and VCAMs, any agent which facilitates the homing process, vascular endothelial growth factor (VEGF), fibroblast growth factors (e.g., FGF4, FGF8, bFGF), Wnt11, DKK1, ascorbic acid, isoproterenol, endothelin, any agent which promotes angiogenesis, including VEGF, aFGF, angiogenin, angiotensin-1 and -2, betacellulin, bFGF, Factor X and Xa, HB-EGF, PDGF, angiomodulin, angiotropin, angiopoietin-1, prostaglandin E1 and E2, steroids, heparin, 1-butyryl-glycerol, and nicotinic amide, any agent which decreases apoptosis including, but not limited to, β-blockers, angiotensin-converting enzyme inhibitors (ACE inhibitors), carvedilol, angiotensin II type 1 receptor antagonists, caspase inhibitors, cariporide, eniporide or a combination thereof.

Lipids and lipid carriers that can be used in culture include: cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

Cells in culture can be maintained either in suspension or attached to a solid support, such as extracellular matrix components and synthetic or biopolymers. Stem cells often require additional factors that encourage their attachment to a solid support, such as type I, type II, and type IV collagen, concanavalin A, chondroitin sulfate, fibronectin, “superfibronectin” and fibronectin-like polymers, gelatin, laminin, poly-D and poly-L-lysine, thrombospondin, and vitronectin.

### Routes of Administration

Dosages employed in practicing the methods of the present disclosure will of course vary depending, e.g., on the particular disease or condition to be treated, the particular natural ERK modulating compound used, the mode of administration, and the therapy desired. natural ERK modulating compound may be administered by any suitable route, including orally, topically (e.g., on the gums), injection, or implantation.

Compositions comprising natural ERK modulating compound may be prepared using conventional diluents or excipients and techniques known in the galenic art. Dosage forms may include tablets, capsules, solutions, suspensions and the like.

### EXAMPLES

### Example 1—Identification of Novel ERK Natural Modulators—Helps Periodontal Ligament Stem Cells Proliferation

To examine whether ERK activator can elevate PDLSC function through Wnt signaling, five different ERK activators are used to treat PDLSCs, and BMSCs (“Bone marrow-derived mesenchymal stem/stromal cells”) serve as the control in this experiment. BrdU analysis demonstrates that PEITC, t-Butylhydroquinone, and resveratrol treatment significantly increases PDLSC proliferation rate, while surprisingly the effect is not observable in BMSCs.

Subsequently, ERK downstream signaling pathways involve screening using a Western blot technique. This includes screening for Shp2 signaling, Notch signaling, and Wnt signaling. Western blot analysis demonstrates that Wnt/β-catenin is highly activated in PDLSCs after ERK activators, PEITC, t-Butylhydroquinone, and resveratrol treatments. Western blot assay further confirms that ERK activator treatment significantly increases the expression level of activated β-catenin. Taken together, and without being bound by theory, these data suggest that ERK activators can increase PDLSC proliferation through activation of Wnt/β-catenin signaling.

### Example 2—Natural ERK Activators Promote Osteogenesis of PDLSCs

To examine the effect of natural compounds PEITC and resveratrol in PDLSC osteogenesis, cells are treated with 1 uM PEITC or 5 uM resveratrol under osteogenic induction. Alizarin red staining shows either PEITC or resveratrol treatment elevate osteogenic differentiation, as indicated by increases in mineralized nodule formation. The stained positive areas are then quantifiable using NIH ImageJ software and shown as a percentage of the total area.

TABLE 1

| Sample | Mineralized Area (% Total Area) |
|---|---|
| Control | 47% ± 3.61 |
| PEITC 1 μM | 65% ± 3.46 |
| Resveratrol 5 μM | 59% ± 2.00 |

The expression of the osteogenic genes runt-related transcription factor 2 (Runx2) and alkaline phosphatase (ALP) are also elevated in PEITC or resveratrol treated groups, assessable by Western blot. Taken together, and without being bound by theory, the data suggest that natural compounds PEITC and resveratrol promote PDLSC osteogenesis through activation of ERK/Wnt pathways.

### Example 3—Natural ERK Activators Promote Immunomodulation of PDLSCs

PEITC and resveratrol are tested to determine whether they contribute to PDLSC immunomodulation. A PDLSC/T cell co-culture experiment examines the immunomodulatory properties of PDLSCs with different compounds treatment. Flow cytometric analysis demonstrates that PDLSCs increases the capacity to induce AnnexinV⁺7AAD⁺ double positive apoptotic CD3⁺ T cells. PEITC or resveratrol treatment significantly promotes PDLSC immunomodulation as indicated by elevated AnnexinV⁺7AAD⁺ double positive apoptotic CD3⁺ T cells.

### Example 4—Natural ERK Activators Protect PDLSCs from Activated T Cell Attack

After co-culture, toluidine blue staining allows for observation of the surviving PDLSCs that remain on the culture plates as positive staining. The results show that activated T cells are able to induce part of PDLSC death in the co-culture system. Natural ERK activators, PEITC and resveratrol, protect PDLSCs survival. Collectively, without being bound by theory, the data suggest that natural ERK activators PEITC and resveratrol surprisingly not only promote PDLSC osteogenesis ability, but also increase PDLSC immunomodulatory function and protect PDLSCs from activated T cell attack.

### Example 5—Curcumin Stimulates Proliferation of GMSC and Periodontal Ligament Stem Cells Differentiation

To examine whether curcumin can elevate the function of GMSCs and PDLSCs, two curcumin extracts, yellow and white, are used to treat GMSCs and PDLSCs with three different concentrations 1, 5, and 10 uM. Stem cell proliferation capabilities are examined by MTT assay. The results show that 1 and 5 uM treatments of both yellow and white curcumin extracts significantly increase GMSC and PDLSC proliferation rate, while 10 uM of yellow and white curcumin extracts show cellular toxicity in GMSCs and PDLSCs.

TABLE 2a
Yellow Curcumin

| Yellow Curcumin Concentration | MTT Assay |
|---|---|
| Control | 0.299 ± 0.032 |
| 1 μM | 0.392 ± 0.078 |
| 5 μM | 0.404 ± 0.081 |
| 10 μM | 0.254 ± 0.041 |

TABLE 2b
White Curcumin

| White Curcumin Concentration | MTT Assay |
|---|---|
| Control | 0.318 ± 0.036 |
| 1 μM | 0.462 ± 0.091 |
| 5 μM | 0.490 ± 0.077 |
| 10 μM | 0.224 ± 0.043 |

### Example 6—Yellow Curcumin Stimulates Differentiation of Periodontal Ligament Stem Cells to Osteogenic Differentiation

To examine the effect of natural compounds yellow and white curcumin extracts in GMSC and PDLSC osteogenesis, cells are treated with 5 μM yellow and white curcumin extracts under osteogenic induction. Alizarin red staining show yellow curcumin extract, but not white curcumin extract treatment elevated osteogenic differentiation, as indicated by increased mineralized nodule formation. The stained positive areas were quantified using NIH ImageJ software and shown as a percentage of the total area. The expression of the osteogenic genes runt-related transcription factor 2 (Runx2) and alkaline phosphatase (ALP) are significantly elevated in yellow curcumin extract, but not white curcumin extract treated groups, assessed by Western blot.

### Example 7—BrdU Analysis of GMSC Proliferation Rate

BrdU analysis demonstrates that PEITC and resveratrol treatment significantly increases GMSC proliferation rate. BrdU incorporation assay demonstrates that treatment of ERK activators, PEITC (1 uM) and resveratrol (5 uM) significantly elevated GMSC cell proliferation.

| Sample | Brdu + BMSC/Total Cells (%) |
|---|---|
| Control | 28.1% ± 0.75 |
| PEITC 1 μM | 48.6% ± 4.79 |
| Resveratrol 5 μM | 47.6% ± 3.17 |

1. A method of treatment of gingiva and/or periodontal ligament tissue to increase dental osteogenesis, mineralization or soft tissue regeneration in the oral cavity, wherein the method comprises administering an effective amount of one or more natural ERK activating compounds to the oral cavity of a patient in need thereof,
wherein the patient has existing endogenous stem cells that are present in the oral cavity, and;
wherein the natural ERK activating compound is administered to the endogenous stem cells in the oral cavity of the patient.

2. The method of claim 1, wherein the natural ERK activating compound is administered to endogenous periodontal ligament stem cells in the patient's oral cavity.

3. The method of claim 1, wherein the natural ERK activating is administered in an amount effective to increase proliferation of endogenous periodontal ligament (PDL) stem cells or gingiva-derived mesenchymal stem cells (GMSC) in the patient's oral cavity.

4. The method of claim 1, wherein the natural compound modulates gene expression or protein expression of a component of the ERK/MAP kinase pathway.

5. The method of claim 1, wherein the natural ERK activating compound can modulate the level of expression of one or more of the following: cellular alkaline phosphatase (ALP), Runx2, bone marrow stromal cells, CD166, CD90, CD105, Stro-1, ATF4, LRP5, TGFβ, osteopontin (OPN), FAS, FASL, and osteocalcin (OCN).

6. The method of claim 1, wherein the natural ERK activating compound is a stilbenoid, a pentacylic terpenoid, a curcuminoid, or an isothiocyanate.

7. The method of claim 6, wherein the natural ERK activating compound is selected from the group consisting of: resveratrol, boswellic acid, curcumin and Phenethyl isothiocyanate (PEITC)

8. The method of claim 1, wherein the amount of the natural compound is effective to increase the proliferation of endogenous PDL stem cells.

9. The method of claim 1, wherein the patient is at elevated risk, relative to a reference standard, of periodontal ligament or gingiva tissue damage.

10. The method of claim 1, wherein the natural ERK activating compound is administered to endogenous gingiva-derived mesenchymal stem cells (GMSC) in the patient's oral cavity.

11. A method of selecting natural ERK activating compounds that cause the proliferation of endogenous oral stem cells wherein the method comprises:
(1) identifying natural ERK activating test compounds by observing increasing in mineralization relative to and a negative control, and/or gene expression of osteogenic biomarkers relative to a positive control and a negative control;
(2) isolating periodontal ligament stem cell cells and/or or gingiva-derived mesenchymal stem cells from an oral cavity,
(3) identifying periodontal ligament stem cells and/or or gingiva-derived mesenchymal stem cells,
(4) contacting and screening periodontal ligament stem cells and/or or gingiva-derived mesenchymal stem cells with identified natural ERK activating test compounds;
(5) determining whether a test compound results in the proliferation of periodontal ligament stem cells and/or or gingiva-derived mesenchymal stem cells; and
(6) selecting a test compound for further development on the basis of whether it increases proliferation of periodontal ligament stem cells and/or or gingiva-derived mesenchymal stem cells relative to a standard control.

12. The method of claim 11, wherein the method is performed as part of a high-throughput method.

13. The method of claim 11, wherein the gene expression or protein expression of a particular cellular marker for osteogenesis is evaluated.

14. The method of claim 13, wherein the level of one or more of the following biomarkers is tested: cellular alkaline phosphatase (ALP), Runx2, bone marrow stromal cells, CD166, CD90, CD105, Stro-1, ATF4, LRP5, TGFβ, osteopontin (OPN), FAS, FASL, and osteocalcin (OCN).

15. The method of claim 11, wherein the method further comprises determining the effect of the natural test compound of interest on bone formation and mineralization in cell culture (in vitro).

16. The method of claim 11, wherein the PDL stem cells are identified by flow cytometry or ELISA.

17. The method of claim 11, wherein a test compound is selected for further development because it increased PDL proliferation and/or or gingiva-derived mesenchymal stem cells in cell culture or an animal model.

18. The method of claim 11, wherein a test compound is identified by its ability to modulate the gene expression or protein expression of a component of the ERK pathway.

19. The method of claim 18, wherein the biomarker is selected from: RSK, MNK, eIF4E, JUN, Fos, SRF, CREB, ATF1, Histone H3, HMG-14, Elk-1, Myc, Max, BRF1, UBF, PP1, PP2A, Akt, Raf, Raf inhibitors, Src, and PAK.

20. The method of claim 1, wherein the natural compound selected for further development is selected from the group consisting of: resveratrol, boswellic acid, curcumin or an extract of curcumin, and Phenethyl isothiocyanate (PEITC)
